(19) United States
(12) Patent Application Publication
(10) Pub. No.: US 2013/0281980 A1
(43) Pub. Date: Oct. 24, 2013

(54) MEDICAL DEVICE FOR ANCHORING A GUIDEWIRE DURING A PERCUTANEOUS CORONARY INTERVENTION

(71) Applicant: Ibrahim R. Al-Rashdan, Safat (KW)

(72) Inventor: Ibrahim R. Al-Rashdan, Safat (KW)

(21) Appl. No.: 13/921,307

(22) Filed: Jun. 19, 2013

Related U.S. Application Data
(62) Division of application No. 12/773,240, filed on May 4, 2010, now abandoned.

(51) Int. Cl. A61M 25/09

(52) U.S. Cl. CPC A61M 25/09
USPC 604/510; 604/528

(57) ABSTRACT

A medical device for anchoring a distal end of a guidewire in a catheter within a coronary artery during a Percutaneous Coronary Intervention including a catheter for being introduced into a coronary artery and a guidewire extending through the catheter. A first anchor device is provided at the proximal end of the guidewire outside of a patient's body and a second anchor is provided at the distal end of the guidewire and catheter to facilitate the movement of interventional devices in the body.

### FIELD OF THE INVENTION

This invention relates to a medical device and/or method for anchoring a guidewire during a Percutaneous Coronary Intervention and more particularly to a device for holding the wire in a distal vessel without using the vessel as a ground for support and therefore avoiding vessel injuries.

### BACKGROUND FOR THE INVENTION

Percutaneous Coronary Interventions(PCI) are a well known medical procedure and have been in use since as early as 1977. The procedure is commonly used to reduce or eliminate the symptoms of coronary artery disease including angina (chest pain), dyspnea (shortness of breath) on exertion, and congestive heart failure. PCI is also used to abort an acute myocardial infraction and in some specific cases it may reduce mortality.

Percutaneous Coronary Interventions are also referred to as balloon angioplasty and are commonly used for the inflation of a balloon within a coronary artery to crush the plaque into the walls of an artery. While balloon angioplasty is still done as a part of nearly all Percutaneous Coronary Interventions, it is seldom the only procedure performed.

Other procedures during PCIs include implantation of stents, rotational or laser Atherectomy and Brachytherapy (use of radio active source to inhibit restenosis). Angioplasty with stenting is frequently a viable alternative to heart surgery for some forms of coronary artery disease. Angioplasty has been shown to reduce symptoms due to coronary artery disease and to reduce cardiac ischemia.

As reported in Wikipedia, the techniques for Percutaneous Coronary Intervention (PCI) usually involve the following steps performed by an interventional Cardiologist with extensive and specialized training. The procedure usually consists of most of the following steps:
1. Access into the femoral artery in the leg, or less commonly, into the radial artery or brachial artery in the arm is created by a device called an “introducer needle”. This procedure is often termed percutaneous access
2. Once access into the artery is gained, a “sheath introducer” is placed in the opening to keep the artery open and control bleeding.
3. Through the sheath, a long flexible, soft plastic tube called a “guiding catheter” is pushed. The tip of the guiding catheter is placed at the mouth of the coronary artery, the guiding catheter also allows for radio opaque dyes (usually iodine based) to be injected into the coronary artery, so that the diseased state and location can be readily accessed using real-time x-ray visualization.
4. During the x-ray visualization, a cardiologist estimates the size of the coronary artery and selects the type of balloon catheter and coronary guidance wire that will be used during the procedure. Heparin (a blood thinner) or medicine used to prevent the formation of blood clots is given to maintain blood flow.
5. The coronary guidewire, which is an extremely thin wire with a radio-opaque flexible tip, is inserted through the guiding catheter and into the coronary artery. While visualizing again by real-time x-ray imaging, the cardiologist guides the wire through the coronary artery to the site of the stenosis or blockage. The tip of the wire is then passed across the blockage. The cardiologist controls the movement and direction of the guidewire by gently manipulating the end that is outside the patient through twisting of the guidewire.
6. While the guidewire is in place, it now acts as the pathway to the stenosis. The tip of the angioplasty or balloon catheter is hollow and is then inserted at the back of the guidewire, thus the guidewire is now inside of the angioplasty catheter. The angioplasty catheter is gently pushed forward, until the deflated balloon is inside of the blockage.
7. The balloon is then inflated, and it compresses the atheromatous plaque and stretches the artery wall to expand.
8. If an expandable wire mesh tube (stent) was on the balloon, then the stent will be implanted (left behind) to support the new stretched open position of the artery from inside.

During Percutaneous Coronary Intervention (PCI) an interventional cardiologist introduces a guidewire into the coronary artery, passed the diseased segment and subsequently advances interventional devices such as a dilation balloon and/or stent to a target over the wire as the guide wire is free floating in the distal vessel, it does not provide any support for advancing devices over a portentous path in route to the target location.

As a general rule Percutaneous transluminal coronary angioplasty (balloon angioplasty), requires manipulation of a catheter from a position outside of a patient's body through an extended portion of the patient's arterial system to the stenopic site for the purpose of eliminating the obstruction by inflating a balloon. This particular procedure has been performed with increasing frequency over the past years in preference to open heart by-pass surgery when possible.

In a typical angioplasty procedure, a guidewire is transluminal inserted into the brachial or the femoral artery, to be positioned within the stenopic region and followed by a balloon catheter. The cardiologist usually pre-bends the distal tip of the guidewire before insertion and then rotates the wire once it has reached the branch artery to enable the guidewire to enter the branch. If the angle of the bend has to be adjusted the guidewire must be removed, re-bent and reinserted sometimes several times. Particular difficulty is encountered pre-bending where an artery branches at one angle and then sub-branches at a different angle. This procedure is attended by the risk of significant trauma to the arterial lining and in many cases the obstruction can not be reached at all with a guidewire and catheter.

Coronary arteries are torturous, and with many sub-branches and often the obstruction is either located where the diameter of the artery is small or by its very presence the obstruction leaves only a very small opening in which a guidewire and/or catheter can be passed. Consequently the cardiologist often finds it very difficult to maneuver the guidewire or catheter which is typically several feet long from the proximal end.

Steering the pre-bent guidewire is further complicated by the fact that branches project at all different radial angles, thus necessitating rotation of the guidewire to the appropriate degree to enter the desired arterial branch. However, rotation of the distal end of the wire typically lags behind rotation of the proximal control end so that precise rotational control is not possible. Also, friction in the arteries can cause the distal end to rotate in a jerky fashion which can traumatize the vascular intima.

One approach to resolving this type of problem is disclosed in a U.S. Pat. No. 4,998,916 of Hammerslag et al. As disclosed therein, an elongate steerable implement which may be either a sterrable guidewire or catheter for coronary angioplasty applications. A floppy steerable tip or a steering region at the distal end of the implement and a control device at the proximal end are connected by means of a plurality of axially movable deflection wires extending throughout the implement. Manipulation of the control permits deflection of the steering region throughout a full 360° range of motion about the axis of the implement, without axial rotation thereof.

### BRIEF SUMMARY OF THE INVENTION

In essence a medical device for anchoring a guidewire and a catheter during

Percutaneous Coronary Intervention includes a catheter having proximal and distal ends insertable into a coronary artery passed a diseased segment and a guidewire having proximal and distal ends extending through the catheter for advancing an interventional device such as balloon and/or stent through the catheter to a target area. The medical device also includes means for anchoring the proximal end of the guidewire outside of a patient's body and means for anchoring the distal end of the guidewire within the catheter at the distal end thereof. The means for anchoring the distal end of the guidewire in the catheter includes a locking styllet including a screw and an anchoring mechanism.

The guidewire anchor in the distal end is composed of two parts, namely a catheter with a monorail system, an end hole of the catheter accommodating a 0.014 inch diameter guidewire and a side hole located at about 1.5 cm from the end hole. The proximal end of the catheter has a conventional locking rim and screw. The second part is a locking styllet having a smooth blunt end on one side and the styllet and screw and latching end.

The invention will now be described in connection with the accompanying drawings wherein like reference numerals have been used to indicate like parts.

### DESCRIPTION OF THE DRAWINGS

FIG. 1 is a schematic illustration of a conventional anchor on a proximal end of a medical device that includes a catheter and a guidewire;

FIG. 2 a schematic illustration of a conventional catheter for Percutaneous Coronary Intervention; and

FIG. 3 is a schematic illustration of an anchor in the distal end of a catheter in accordance with the present invention.

### DESCRIPTION OF THE PREFERRED EMBODIMENTS OF THE INVENTION

During percutaneous coronary interventions, the interventional cardiologist introduces a guide wire into the coronary artery past the diseased segment, and subsequently interventional devices such as dilation balloon and stents are advanced to the target location for therapy over the wire as a monorail system.

As the guide wire is floating free in the distal vessel it does not provide any support for advancing devices over a tortuous or angulated path in route to the target location. Few interventional techniques are used to facilitate device passage over resistant areas and no device is available to Applicant's knowledge to address this demanding challenge.

During percutaneous coronary interventions, as the proximal part of the guide wire is secured outside of the body, anchoring the guide wire in the distal vessel past the target location will add support for device delivery akin to a tight wire with both of its ends secure. However, in the present case the wire anchor devices promises to hold the wire in the distal vessel without using the vessel as a ground for support and thus avoids injury to the vessel by being free in the vessel lumen.

In essence, the wire anchor is composed of two parts:
1.) The catheter with a monorail system, an end hole of the catheter accommodates a 0.014 inch guide wire and includes a side hole located at approximately 1.5 cm proximal to the end hole and acts as guide wire exit to allow for a monorail system. The proximal part of the catheter has a locking rim and screw adapted as a latch mechanism of the stilet of part 2.
2.) The locking stilet with smoothed blunt end at one end and screw and latch locking end on the other side of the stilet is 0.014 inch in diameter.

The procedure of the wire anchor uses:
1.) The interventional cardiologists use regular equipment for the PCI procedure and after the guide wire introduction if advancement of interventional devices is proved difficult, then the wire anchor is utilized.
2.) Part 1 of the catheter is guided over the same interventional wire to the distal tip of the target vessel and passed the target lesion.
3.) The locking stilet part 2 is advanced through the central hub of the catheter part 1 and locked in place using the screw and latch. This anchors the wire in place.
4.) Then the usual interventional devices are advanced over the guide wire past the resistant areas.
5.) Once the interventional device is in location the wire anchor is unlocked and detached from the guide wire and removed.
6.) At this time the interventional device can be deployed.

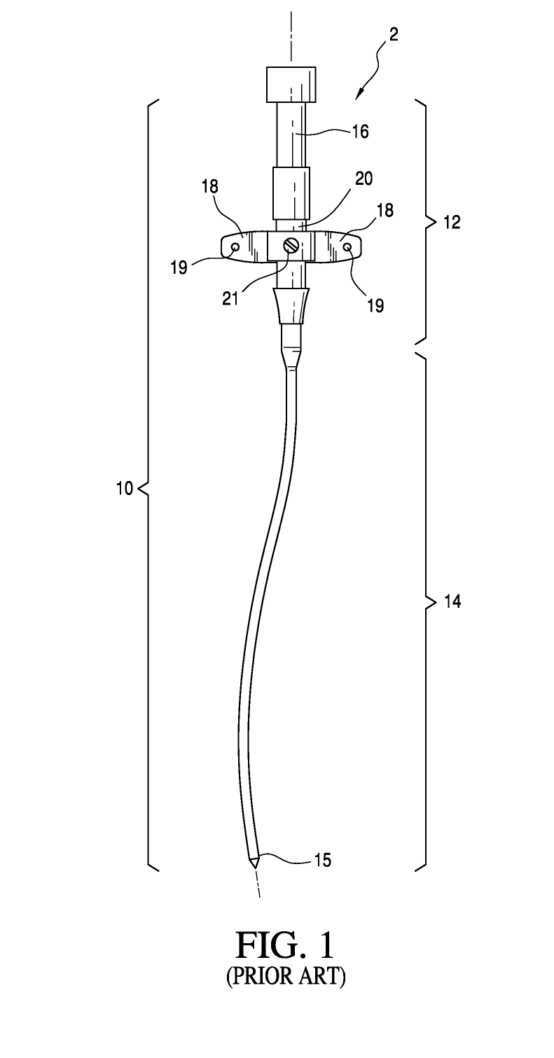

A prior art catheter 2 is illustrated in FIG. 1 wherein a catheter 10 has a proximal end 12 and a distal end 14 which may be used for Percutaneous Coronary Intervention. As shown in FIG. 1, a hub 16 is located at or near an entry site of a patient's body and a suture wing component 18 surrounds a portion of the hub 16 that has a reduced diameter section 20.

The suture wing portion has a first portion with a lumen and the reduced diameter section 20 of the hub 16 is disposed within the lumen such that the lumen and the hub are co-axial with respect to the longitudinal axis that extends to the tip 15. The suture wing component 18 fits in the reduced diameter section of the hub 16, but allows some “play” to allow relative movement both axial and rotational between the hub 16 and suture wing component 18. However, a screw 21 is provided to anchor the catheter 2 or guidewire (not shown in FIG. 1) in the proximal end 12.

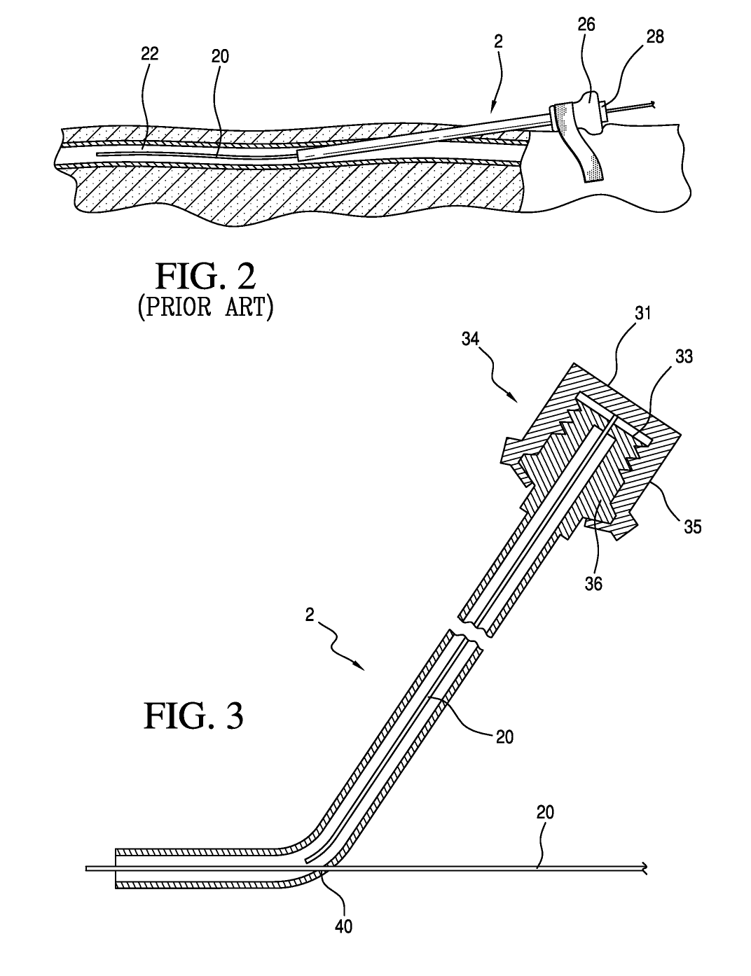

A guidewire 20 as illustrated in FIGS. 2 and 3 extends through the catheter 2 and into a coronary artery 22.

As illustrated in FIG. 2, the catheter 2 includes an anchoring element 26 that is on the outside of a patient's body and attached to the patient's body by tape (not shown). The catheter 2 also includes a locking element 28 that is screwed on to the anchoring element 26 to rigidly fix the guidewire in place within the element 26.

Referring now to FIG. 3, the catheter 2 also includes a distal end 31 that is anchored within a distal end 33 of the catheter 2 by means of an anchoring element 34. The anchoring element 34 includes an outer segment 35 with internal threads and an inner threaded clamp 36 with external threads for securely holding the guidewire 20 within a distal portion of the catheter 2. The catheter 2 also includes a guidewire side hole 40. The latch is unlocked and the stillet removed. Then the catheter is pushed forward to free it from the guidewire and then removed.

During a Percutaneous Coronary Intervention an interventional cardiologist introduces a guidewire into a coronary artery passed the diseased segment and subsequently advances interventional devices such as dilation balloons and stents to a target area for therapy over the guidewire since the guidewire is floating free in the distal vessel artery it does not provide any support for advancing devices over a tortuous or angularly deviating pathway in route to a target location. Few interventional techniques are available to facilitate passage for the interventional devices over resistant areas. During Percutaneous Coronary Interventions, the proximal part of the guidewire is secured outside of the body. Therefore, anchoring the guidewire at the distal vessel part past the target location will add support to the device like a tightrope wire that is fixed at both ends to provide a sort of monorail. Further, fixing the guidewire at the distal end is done without using the vessel as a support and thus avoids injury to a coronary artery.

In making a Percutaneous Coronary Intervention an interventional cardiologist uses a regular catheter and guidewire for the PCI procedure and after the guidewire introduction advances interventional devices such as a balloon or stent. However, when the advancement of the interventional devices is difficult, then the wire anchor in the distal end of the catheter is utilized. In this practice part 1 of the catheter glides over the same guidewire to the distal tip of the guidewire and past the target region. The locking styllet (part 2) is advanced through the central hub of a catheter (part 1) and locked in place using a screw and latch mechanism. This anchors the guidewire in place.

Then the usual interventional devices are advanced over the guidewire passed the resistant area. Then, once the interventional device is in a pre-selected or target area, the wire anchor is unlocked and released and the wire anchor device is removed. At this time, the interventional device is deployed. The guidewire remains in place while the anchor device is removed. The balloon is positioned over the guidewire

The stent is advanced over the guidewire if it meets resistance.

While the invention has been described in connection with its preferred embodiments, it should be recognized that changes and modifications may be made therein without departing from the scope of the appended claims.

1-4. (canceled)

5. A medical device for anchoring a guidewire and a catheter during Percutaneous Coronary Intervention, said device consisting of:
a catheter having proximal and distal ends insertable into a coronary artery past a diseased segment;
a guidewire having proximal and distal ends and a diameter of about 0.014 inch extending through said catheter for advancing an interventional device through said catheter to a target area;
means for anchoring the proximal end of said guidewire outside of a patient's body;
means for anchoring said distal end of said guidewire within said catheter and wherein said means for anchoring said distal end of said guidewire in said catheter includes a locking styllet with a smooth blunt end on one side thereof and a screw and lock mechanism on the other side thereof; and
in which said catheter includes a guidewire exit hole at about 1.5 cm from said proximal end of said catheter.

6. A method for anchoring a distal end of a guidewire in a distal end of a catheter during Percutaneous Coronary Intervention, said method comprising the steps of:
providing a catheter having proximal and distal ends insertable into a coronary artery;
inserting a distal end of said catheter into a patient's coronary artery and advancing said catheter through said artery and past a diseased segment;
providing a guidewire having proximal and distal ends extending through said catheter for advancing an interventional device through said catheter to a target area;
employing the interventional device at the target area; and
removing the anchor from the distal end and the guidewire from the coronary artery and activating the interventional device.
